(19) United States
(12) Plant Patent Application Publication
(10) Pub. No.: US 2003/0033651 P1
(43) Pub. Date: Feb. 13, 2003

(54) Chrysanthemum plant named 'Etna'

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/923,534

(22) Filed: Aug. 8, 2001

(51) Int. Cl. A01H 5/00

(52) U.S. Cl. PLT/292

(57) ABSTRACT

A Chrysanthemum plant named ‘Etna’ characterized by its SMALL sized blooms with PINK ray-florets and A darker pink cushion center.

### BACKGROUND OF THE INVENTION

‘Etna’ is a product of a breeding-program which had the objective of creating new chrysanthemum cultivars with a anemone type flower, a 7 week response and a medium plant height. The new plant of the present invention comprises a new and distinct cultivar of Chrysanthemum plant. ‘Etna’ is a seedling from a cross in a breeding program maintained under the control of inventor. The female parent is U.S.Plant Pat. . . . , not available to inventor for description. The male parent is unknown, being a mixed pollation of a group of male parents. The new and distinct cultivar was discovered and selected as a flowering plant within the progeny of the stated cross by Rob Noodelijk in a controlled environment (greenhouse) in Rijsenhout Holland in August 1998. The first act of asexual reproduction of ‘Etna’ was accomplished when vegetative cuttings were taken from the initial selection in October 1998 in a controlled environment in Rijsenhout Holland.

### SUMMARY OF THE INVETION

The present invention is a new and distinct variety of chrysanthemum bearing SMALL sized blooms with PINK ray-florets and a darker pink cushion center.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

FIG. 2 shows the various stages of bloom of the new cultivar.

FIG. 3 shows the foliage of the new cultivar

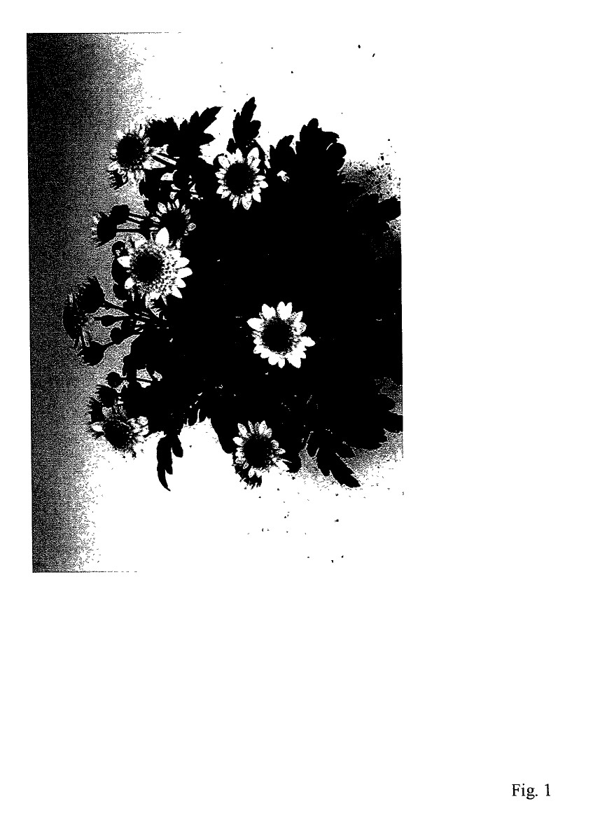

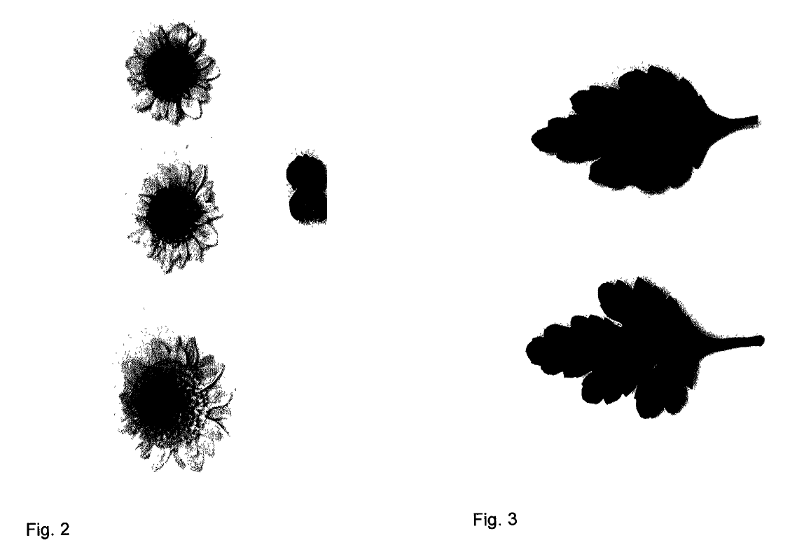

### DESCRIPTION OF THE INVENTION

This new variety of chrysanthemum is of the botanical classification dendranthema grandiflora. The observations and measurements were gathered from plants grown in a greenhouse in Rijsenhout Holland in a photo-periodic controlled crop under conditions generally used in commercial practice. The greenhouse temperatures during this crop were at day-time between 18° C. and 25° C. and at night 20° C. The photo-periodic response time in this crop was 52 days after an average of eight long days. After this long day period to flowering growth retardants were applied 6 times in an average dose of 1.5 gram/liter water. No tests were done on disease or insect resistance or susceptibility. This new variety produces SMALL sized blooms with PINK ray-florets and SCHIJF disc-florets blooming on the plant for 5 weeks. This new variety of chrysanthemum has been found to retain its distinctive characteristics throughout successive propagations however the phenotype may vary significantly with variations in environment such as light intensity and temperature. To show the phenotype as described ‘Etna’ can be planted without assimilation lightning (high pressure sodium lamps) between week 50 and week 40 of the next year under greenhouse conditions in Holland. With assimilation lightning (minimum level 2500 lux) it can be planted year round under greenhouse conditions in Holland.

From the cultivars known to inventor the most similar existing cultivar in comparison to ‘Etna’ is ‘Pixie Time’ (unpatented). Both ‘Etna’ and ‘Pixie Time’ have a bi-color anemone type flower. When ‘Pixie Time’ and ‘Etna’ are being compared the following differences are noticed: The differences of ‘Pixie Time’ and ‘Etna’ are (1) Flower color. The bicolor effect of ‘Etna’ is stronger. (2) Vigor. Etna is more vigorous. (3) Plant habit. The width of ‘Etna’ is larger.

The following is a description of the plant and characteristics that distinguish ‘Etna’ as a new and distinct variety. The color designations are taken from the plant itself. Accordingly, any discrepancies between the color designations and the colors depicted in the photographs are due to photographic tolerances. The color chart used in this description is: The Royal Horticultural Society color chart, edition 1995.

I claim:

1. A new and distinct variety of chrysanthemum plant as described and illustrated.
